(19) United States
(12) Patent Application Publication
(10) Pub. No.: US 2026/0151092 A1
(43) Pub. Date: Jun. 4, 2026

(54) SYSTEMS AND METHODS TO PREDICT GLOBAL HYPOPERFUSION IN CRITICAL CARE PATIENTS

(71) Applicant: Becton, Dickinson and Company, Franklin Lakes, NJ (US)

(72) Inventors: Kevin James Moses, Tustin, CA (US); Cristhian M. Potes Blandon, Rancho Santa Margarita, CA (US); Christine Lee, Long Beach, CA (US)

(21) Appl. No.: 19/458,006

(22) Filed: Jan. 23, 2026

Related U.S. Application Data
(63) Continuation of application No. PCT/US2024/039652, filed on Jul. 25, 2024.
(60) Provisional application No. 63/515,808, filed on Jul. 26, 2023.
(60) Provisional application No. 63/623,048, filed on Jan. 19, 2024.

(51) Int. Cl. A61B 5/00; A61B 5/02; A61B 5/0215; A61B 5/026; A61B 5/1455

(52) U.S. Cl. CPC A61B 5/7239; A61B 5/02007; A61B 5/02028; A61B 5/0215; A61B 5/026; A61B 5/14551; A61B 5/7203; A61B 5/7221; A61B 5/7264; A61B 5/7275; A61B 5/742; A61B 5/746; A61B 5/7475; A61B 2562/227

(57) ABSTRACT

A system for determining a global hypoperfusion index (GHI) includes an arterial blood pressure sensor, a ventricular blood pressure sensor, an oximetry module, and an integrated hardware unit including a system processor and a system memory. The system memory includes instructions that cause the system to receive arterial hemodynamic data, ventricular hemodynamic data, and blood oxygen saturation data. One or more right ventricular pressure features, one or more pulmonary artery pressure features, one or more cardiac output parameters, and one or more venous oxygen saturation parameters are derived from the arterial hemodynamic data, the ventricular hemodynamic data, and the blood oxygen saturation data. The GHI is derived by using a predictive decision model based upon the one or more right ventricular pressure features, the one or more pulmonary artery features, the one or more cardiac output parameters, and the one or more venous oxygen saturation parameters.

### CROSS-REFERENCE TO RELATED APPLICATION(S)

This application is a continuation of PCT Application No. PCT/US2024/039652 filed Jul. 25, 2024, and entitled “SYSTEMS AND METHODS TO PREDICT GLOBAL HYPOPERFUSION IN CRITICAL CARE PATIENTS,” which claims the benefit of U.S. Provisional Application No. 63/515,808, filed Jul. 26, 2023, and entitled “SYSTEM AND METHOD TO PREDICT GLOBAL HYPOPERFUSION IN CRITICAL CARE PATIENTS,” and U.S. Provisional Application No. 63/623,048, filed Jan. 19, 2024, and entitled “SYSTEMS AND METHODS TO PREDICT GLOBAL HYPOPERFUSION IN CRITICAL CARE PATIENTS,” the disclosures of which are hereby incorporated by reference in their entireties.

### BACKGROUND

The present disclosure relates to hemodynamic monitoring and, in particular, to predicting global hypoperfusion of a patient.

Global hypoperfusion describes the inadequate oxygen delivery to meet metabolic demands. A hypoperfusion event can be triggered by various physiological responses in the body, including problems with pulmonary system that result in reduction of oxygen supply, problems with the delivery of oxygen to the cells of the body (e.g., insufficient cardiac output (CO), low hemoglobin count, and/or bleeding events) or a sudden increase in oxygen demand. A global hypoperfusion event in a patient can result in serious harm and thus, a method of predicting when a global hypoperfusion event will occur is desirable.

### SUMMARY

A system for determining a global hypoperfusion index (GHI) representative of a prediction of a future global hypoperfusion event within a patient. The system includes an arterial blood pressure sensor including a housing, a fluid input port connected via tubing to a fluid source, a catheter-side fluid port connected to a catheter inserted within an arterial system of a patient, a pressure transducer in communication with the fluid source through the fluid port, and an I/O cable in electrical communication with the pressure transducer. The system further includes a ventricular blood pressure sensor including a housing, a fluid input port connected via tubing to a fluid source, a catheter-side fluid port connected to a catheter inserted within a ventricular system of a patient, a pressure transducer in communication with the fluid source through the fluid port, and an I/O cable in electrical communication with the pressure transducer. The system further includes an oximetry module including an optical transmitter, an optical receiver, and an I/O cable in electrical communication with the optical transmitter and the optical receiver. The system also includes an integrated hardware unit including a system processor, a system memory, a display including a user interface, and an analog-to-digital (ADC) converter. The system memory includes instructions that, when executed by the system processor, cause the system to receive arterial hemodynamic data from the arterial blood pressure sensor, receive ventricular hemodynamic data from the ventricular blood pressure sensor, and receive a blood oxygen saturation data from the oximetry module. One or more right ventricular pressure features are derived, using a first algorithm, from the ventricular hemodynamic data. One or more pulmonary artery pressure features are derived, using a second algorithm, from the arterial hemodynamic data. One or more cardiac output parameters are derived, using a third algorithm, from the ventricular hemodynamic data and/or the arterial hemodynamic data. One or more venous oxygen saturation parameters are derived, using a fourth algorithm, from the blood oxygen saturation data. The GHI is derived by using a predictive decision model based upon the one or more right ventricular pressure features, the one or more pulmonary artery features, the one or more cardiac output parameters, and the one or more venous oxygen saturation parameters. The GHI is displayed on the display.

A method for determining a global hypoperfusion index (GHI) representative of a prediction of a future global hypoperfusion event within a patient includes receiving a plurality of hemodynamic data from arterial and ventricular blood pressure sensors, receiving a blood oxygen saturation data from an oximetry module, deriving, using a first algorithm, one or more right ventricular pressure features from the plurality of right ventricular hemodynamic data, deriving, using a second algorithm, one or more pulmonary artery pressure features from the plurality of pulmonary arterial hemodynamic data, deriving, using a third algorithm, one or more cardiac output parameters from the plurality of hemodynamic data, deriving, using a fourth algorithm, one or more venous oxygen saturation parameters from the blood oxygen saturation data, deriving the GHI by using a predictive decision model based upon the one or more right ventricular pressure features, the one or more pulmonary artery features, the one or more cardiac output parameters, and the one or more venous oxygen saturation parameters, and displaying the GHI on a hemodynamic display.

### BRIEF DESCRIPTION OF THE DRAWINGS

FIG. 1 is a schematic block diagram of a hemodynamic monitoring system for generating a global hypoperfusion index (GHI).

FIG. 2 is a perspective view of an example hemodynamic monitor.

FIG. 3 is a perspective view of an example catheter that can be inserted in the patient and connected to one or more hemodynamic sensors for providing hemodynamic data to the hemodynamic monitor.

FIG. 4 is a perspective view of an example minimally invasive pressure sensor that can be attached to the patient for sensing hemodynamic data representative of a right ventricular pressure or a pulmonary artery pressure of the patient.

FIG. 5 is a perspective view of an example oximetry module for receiving oximetry data from a catheter inserted within the patient.

FIG. 6 is a block diagram of a GHI algorithm.

FIG. 7 is a graph illustrating an example right ventricular pressure (RVP) waveform trace including example indicia indicative of blood flow, cardiac output, and global hypoperfusion.

FIG. 8 is a graph illustrating an example pulmonary artery pressure (PAP) waveform trace including example indicia indicative of blood flow, cardiac output, and global hypoperfusion.

FIG. 9 is a diagram of an example of a system for implementing a right ventricular cardiac output (RVCO) algorithm.

FIG. 10 is a diagram of an alternative example of the system for implementing an (RVCO) algorithm.

FIG. 11 is a table showing an example of an output of the system for generating the GHI.

FIG. 12 is a method flow diagram illustrating a method for generating the GHI.

### DETAILED DESCRIPTION

As described herein, a system for determining a global hypoperfusion index (GHI) is used to predict a future global hypoperfusion event in a patient. The system can determine a real-time, continuous GHI that predicts a likelihood of a hypoperfusion event. The system for determining a GHI includes a pulmonary catheter. The pulmonary catheter includes a plurality of hemodynamic sensors configured to measure oximetry data, cardiac output data, pulmonary artery pressure data, and right ventricular pressure data. A processor uses a plurality of algorithms, described in more detail herein, to derive features and values from the oximetry data, pulmonary artery pressure data, and right ventricular pressure data. The processor then executes a machine learning algorithm based upon the features and values derived from the plurality of algorithms in order to generate the GHI. The GHI is then transmitted to an output system, where it is then viewable, for example, by a clinician.

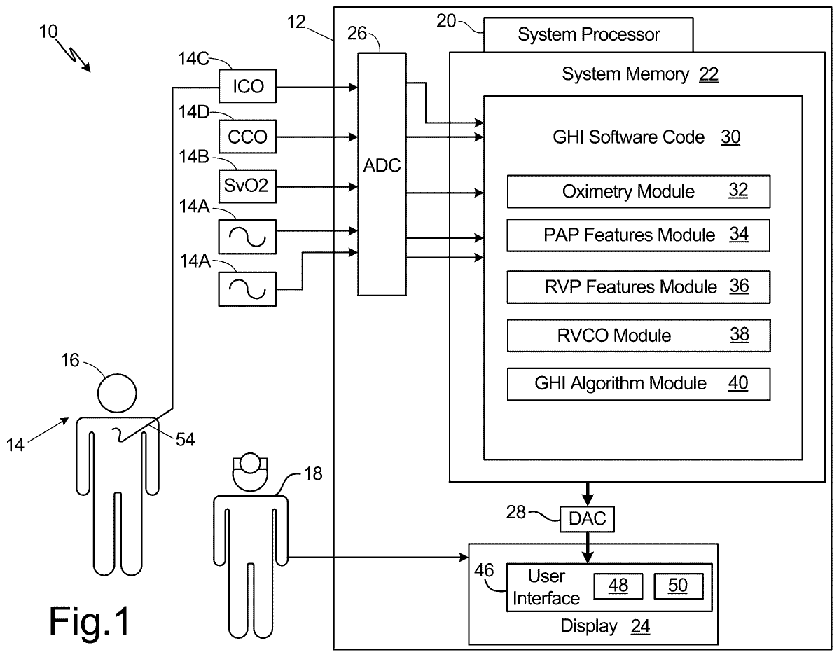

FIG. 1 is a schematic block diagram of hemodynamic monitoring system 10 for generating a global hypoperfusion index (GHI). Hemodynamic monitoring system 10 includes hemodynamic monitor 12 and hemodynamic sensors 14 (including hemodynamic sensors 14A, 14B, 14C, and 14D). Hemodynamic monitor 12 includes system processor 20, system memory 22, display 24, analog-to-digital converter (ADC) 26, and digital-to-analog convertor (DAC) 28. System memory 22 includes GHI software code 30, which includes oximetry module 32, pulmonary artery pressure (PAP) features module 34, right ventricular pressure (RVP) features module 36, right ventricular cardiac output (RVCO) module 38, and GHI algorithm module 40. Display 24 includes user interface 46, which includes control elements 48 and sensory alarm 50. FIG. 1 also shows patient 16, healthcare worker 18, and catheter 54.

As illustrated in FIG. 1, hemodynamic monitoring system 10 includes hemodynamic monitor 12 and hemodynamic sensors 14 (including hemodynamic sensors 14A, 14B, 14C, and 14D). Hemodynamic monitoring system 10 can be implemented within a patient care environment, such as an ICU, an OR, or other patient care environment, for monitoring a hemodynamic condition of a patient. As illustrated in FIG. 1, the patient care environment can include patient 16 and healthcare worker 18 trained to utilize hemodynamic monitoring system 10.

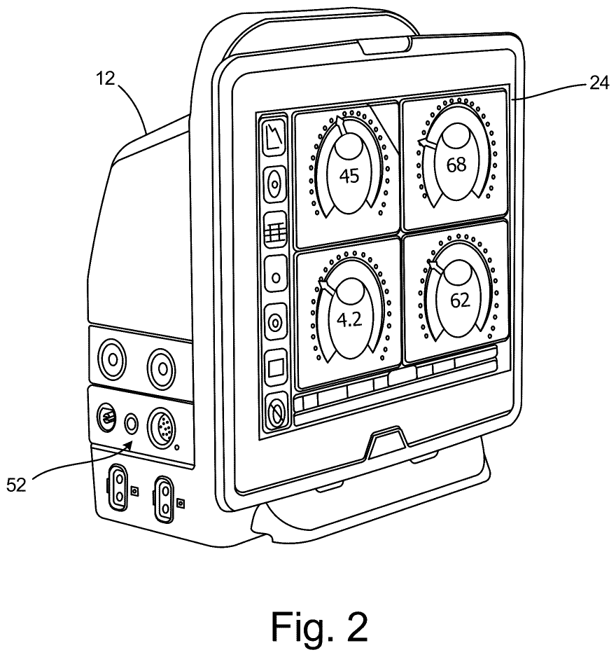

Hemodynamic monitor 12, as described below with respect to FIG. 2, can be an integrated hardware unit that includes system processor 20, system memory 22, display 24, ADC 26, and DAC 28. In other examples, any one or more components and/or described functionality of hemodynamic monitor 12 can be distributed among multiple hardware units. For instance, in some examples, display 24 can be a separate display device that is remote from and operatively coupled with hemodynamic monitor 12. Likewise, at least a portion of data processing within hemodynamic monitoring system 10 can occur via a smart cable that is connected between a catheter (e.g., catheter 54) or sensor and hemodynamic monitor 12. In general, though illustrated and described in the example of FIG. 1 as an integrated hardware unit, it should be understood that hemodynamic monitor 12 can include any combination of devices and components that are electrically, communicatively, or otherwise operatively connected to perform functionality attributed herein to hemodynamic monitor 12.

As illustrated in FIG. 1, system memory 22 stores GHI software code 30. GHI software code 30 includes oximetry module 32, PAP features module 34, RVP features module 36, RVCO module 38, and GHI algorithm module 40. Display 24 provides user interface 46, which includes control elements 48 that enable user interaction with hemodynamic monitor 12 and/or other components of hemodynamic monitoring system 10. User interface 46, as illustrated in FIG. 1, also provides sensory alarm 50 to provide warning to medical personnel based on the hemodynamic status of patient 16, as is further described below.

Hemodynamic sensors 14 can be attached to patient 16 to sense hemodynamic data representative of an RVP waveform, a PAP waveform, blood oxygen saturation (labeled “SvO2” in FIG. 1), or cardiac output of patient 16 or any combination of these hemodynamic data. Hemodynamic sensors 14 are operatively connected to hemodynamic monitor 12 (e.g., electrically and/or communicatively connected via wired or wireless connection, or both) to provide the sensed hemodynamic data to hemodynamic monitor 12. In some examples, hemodynamic sensors 14 provide the hemodynamic data of patient 16 to hemodynamic monitor 12 as an analog signal, which is converted by ADC 26 to digital hemodynamic data representative of the RVP waveform and/or the PAP waveform. In other examples, hemodynamic sensors 14 can provide the sensed hemodynamic data to hemodynamic monitor 12 in digital form, in which case hemodynamic monitor 12 may not include or utilize ADC 26. In yet other examples, hemodynamic sensors 14 can provide the hemodynamic data of patient 16 to hemodynamic monitor 12 as an analog signal, which is analyzed in its analog form by hemodynamic monitor 12.

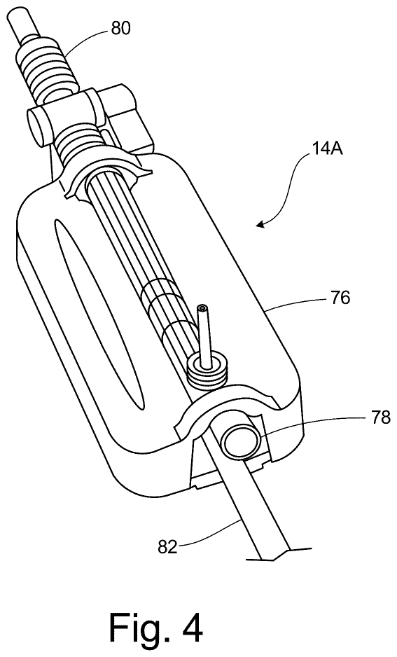

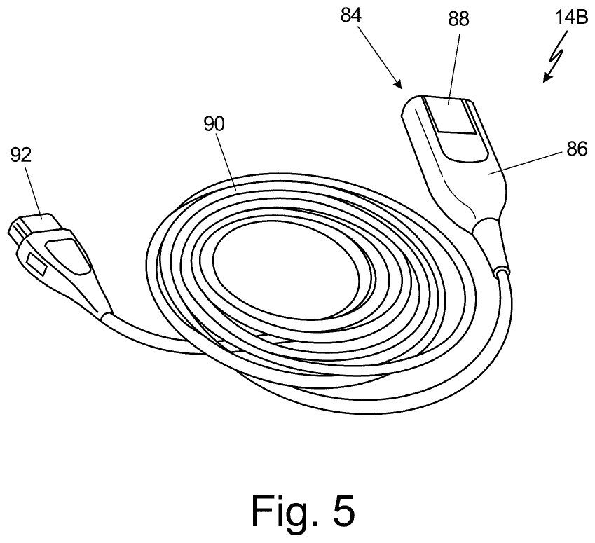

Hemodynamic sensors 14 can include one or more non-invasive, minimally invasive, or invasive sensors attached to patient 16. For instance, hemodynamic sensors 14 can take the form of invasive hemodynamic sensor 14A, such as second pressure transducer 14A that provides RVP waveform data sensed at right ventricle port 68C located within the right ventricle of the patient's heart (shown in FIG. 4). Hemodynamic sensors 14 can take the form of invasive hemodynamic sensor 14B, such as oximetry module 14B that provides blood oxygen saturation data within the pulmonary artery based on light pulses emitted from module 14B into the pulmonary artery and reflected, returned, and received by module 14B via optical connector 62 (shown in FIG. 5) of catheter 54. In yet other examples, hemodynamic sensors 14 can take the form of non-invasive hemodynamic sensors. In some examples, hemodynamic sensors 14 can be attached non-invasively at an extremity of patient 16, such as a forehead, a wrist, an arm, a finger, an ankle, a toe, or other extremity of patient 16. Hemodynamic sensors 14 can also take the form of other invasive, minimally invasive, or non-invasive hemodynamic sensors.

In certain examples, hemodynamic sensors 14 can be configured to sense RVP, PAP, or both right ventricular and pulmonary artery pressures of patient 16. In some instances, hemodynamic sensors 14 may also be used to sense cardiac output of the patient, blood oxygen saturation within the pulmonary artery, or both cardiac output and blood oxygen saturations in addition to right ventricular and pulmonary artery pressures. For instance, one or more hemodynamic sensors 14 can be attached to patient 16 via a radial arterial catheter inserted into an arm of patient 16. In other examples, one or more hemodynamic sensors 14 can be attached to patient 16 via a femoral arterial catheter inserted into a leg of patient 16. Such techniques can similarly enable multiple hemodynamic sensors 14 to provide substantially continuous beat-to-beat monitoring of the RVP and PAP as well as monitoring of cardiac output, and blood oxygen saturation of patient 16, or any combination of these hemodynamic data, over an extended period of time, such as minutes or hours.

System processor 20 executes GHI software code 30, which implements oximetry module 32, PAP features module 34, RVP features module 36, RVCO module 38, and GHI algorithm module 40, which utilize the RVP waveform, the PAP waveform, and the blood oxygen saturation data to determine global hypoperfusion for patient 16.

Processor 20, in some examples, is configured to implement functionality and/or process instructions for execution within system 10. For instance, processor 20 can be capable of processing instructions stored in system memory 22. Examples of processor 20 can include any one or more of a microprocessor, a controller, a digital signal processor (DSP), an application specific integrated circuit (ASIC), a field-programmable gate array (FPGA), or other equivalent discrete or integrated logic circuitry.

System memory 22 can be configured to store information within hemodynamic monitor 12 during operation. System memory 22, in some examples, is described as computer-readable storage media. In some examples, a computer-readable storage medium includes a non-transitory medium. The term “non-transitory” indicates that the storage medium is not embodied in a carrier wave or a propagated signal. In certain examples, a non-transitory storage medium stores data that, over time, changes (e.g., in RAM or cache). In some examples, system memory 22 is a temporary memory, meaning that a primary purpose of system memory 22 is not long-term storage. System memory 22, in some examples, is described as volatile memory, meaning that system memory 22 does not maintain stored contents when electrical power to system memory 22 is removed. Examples of volatile memories can include random access memories (RAM), dynamic random access memories (DRAM), static random access memories (SRAM), and other forms of volatile memories. In some examples, system memory 22 is used to store program instructions for execution by processor 20. System memory 22, in one example, is used by software or applications to temporarily store information during program execution.

System memory 22, in some examples, also includes one or more computer-readable storage media. System memory 22 is configured to store larger amounts of information than volatile memory. System memory 22 is further configured for long-term storage of information. In some examples, system memory 22 includes non-volatile storage elements. Examples of such non-volatile storage elements include, but are not limited to, magnetic hard discs, optical discs, flash memories, or forms of electrically programmable memories (EPROM) or electrically erasable and programmable (EEPROM) memories.

Display 24 can be a liquid crystal display (LCD), a light-emitting diode (LED) display, an organic light-emitting diode (OLED) display, or other display device suitable for providing information to users in graphical form. User interface 46 can include graphical and/or physical control elements that enable user input to interact with hemodynamic monitor 12 and/or other components of hemodynamic monitoring system 10. In some examples, user interface 46 can take the form of a graphical user interface (GUI) that presents graphical control elements presented at, e.g., a touch-sensitive and/or presence sensitive display screen of display 24. In such examples, user input can be received in the form of gesture input, such as touch gestures, scroll gestures, zoom gestures, or other gesture input. In certain examples, user interface 46 can take the form of and/or include physical control elements, such as a physical buttons, keys, knobs, or other physical control elements configured to receive user input to interact with components of hemodynamic monitoring system 10.

In operation, one or more hemodynamic sensors 14 are connected to hemodynamic monitor 12 and catheter 54. Hemodynamic sensor 14A senses hemodynamic data representative of an right ventricular pressure (RVP) waveform, a pulmonary artery pressure (PAP) waveform, and/or another blood pressure waveform of patient 16. Hemodynamic sensor 14A provides the hemodynamic data (e.g., as analog sensor data) to hemodynamic monitor 12. ADC 26 converts the analog hemodynamic data to digital hemodynamic data representative of the RVP waveform, the PAP waveform, and/or another blood pressure waveform of patient 16. Hemodynamic sensor 14B sensors hemodynamic data representative of mixed venous oxygen saturation (SvO2) value.

In operation, system memory 22 is encoded with instructions that are executed by processor 20. System memory 22 includes oximetry module 32. Oximetry module 32 includes one or more programs containing instructions to apply an SvO2 algorithm to the oximetry data received from catheter 54, and more specifically from, for example, hemodynamic sensor 14B. Upon execution of oximetry module 32, oximetry data (i.e., transmitted from catheter 54) is processed via the SvO2 algorithm to produce one or more SvO2 parameters. In some examples, the SvO2 parameters can include a measured SvO2 value and a signal quality index (SQI).

The measured SvO2 parameter is a measure of the oxygen content of the blood returning to the heart after perfusing through the body. An abnormal SvO2 can be indicative of inadequate systemic oxygenation. The SvO2 can be sampled from hemodynamic sensor 14B such that a new SvO2 value is measured on a defined time interval (e.g., the SvO2 is sampled once every 2 seconds). The SQI can be indicative of the overall reliability and readability of the received oximetry data. Thus, a noisier signal would give rise to a lower SQI indicating lower levels of signal reliability, and a less noisy signal would give rise to a higher SQI indicating higher levels of signal reliability. In some examples, the SQI is presented via a numerical scale (e.g., 0-5, wherein 0 is the lowest quality and 5 is the highest quality). In some examples, GHI software code 30 only accepts the signal for further processing if the SQI is above a defined threshold (e.g., SQI is greater than 1, and thus between 2-5).

System memory 22 further includes PAP features module 34. PAP features module 34 includes one or more programs containing instructions to apply a PAP algorithm to the PAP waveform received from catheter 54, and more specifically from hemodynamic sensor 14A. Upon execution of PAP features module 34, the PAP waveform (i.e., transmitted from catheter 54) is processed via the PAP algorithm to produce one or more PAP features. In some examples, the PAP features can include a mean PAP value. The mean PAP value can be a mean value of the PAP waveform for a defined time interval. For example, the mean PAP can be determined for a 10-second window of the PAP waveform. In other examples, the mean PAP is determined for an individual heartbeat, where the start and end of an individual heartbeat within the PAP waveform is determined by a beat detection algorithm. Other PAP features can also be determined by PAP features module 118, and are discussed in greater detail in the description of FIG. 8.

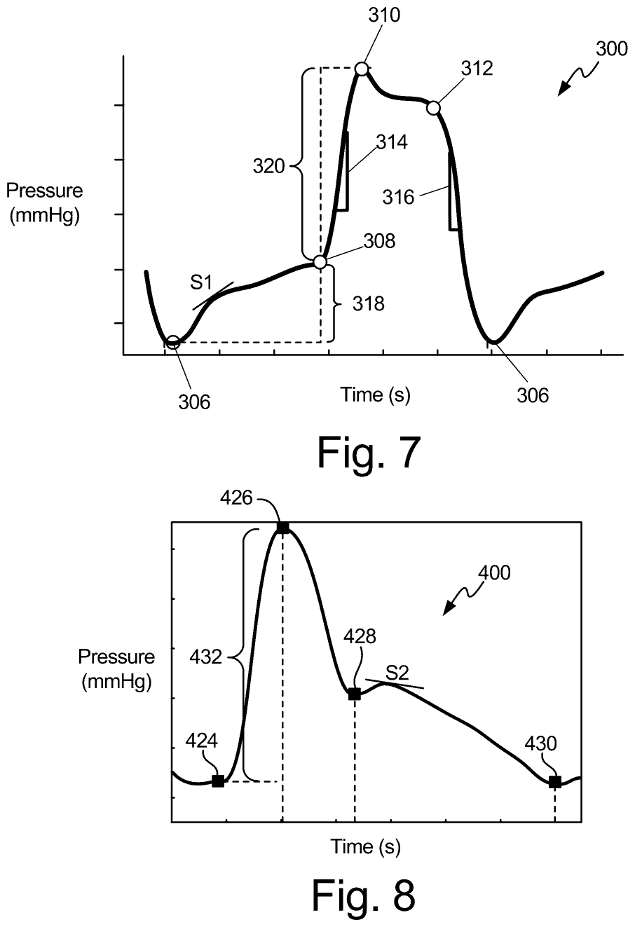

System memory 22 further includes RVP features module 36. RVP features module 36 includes one or more programs containing instructions to apply an RVP algorithm to the RVP waveform received from catheter 54, and more specifically from hemodynamic sensor 14A. Upon execution of RVP features module 36, the RVP waveform (transmitted from catheter 54) is processed via the RVP algorithm to produce one or more RVP features. In some examples, the RVP features can include one or more of: a pulse rate, a maximum pressure rate of change with respect to time (“dP/dt”) during systolic rise, a minimum dP/dt during a relaxation period after end systole, a systole time, a systolic pressure, an end systolic pressure, an end diastolic pressure, a pulse pressure, and a mean right ventricular pressure. The RVP features and their derivation from the RVP waveform are discussed in greater detail in the description of FIG. 7.

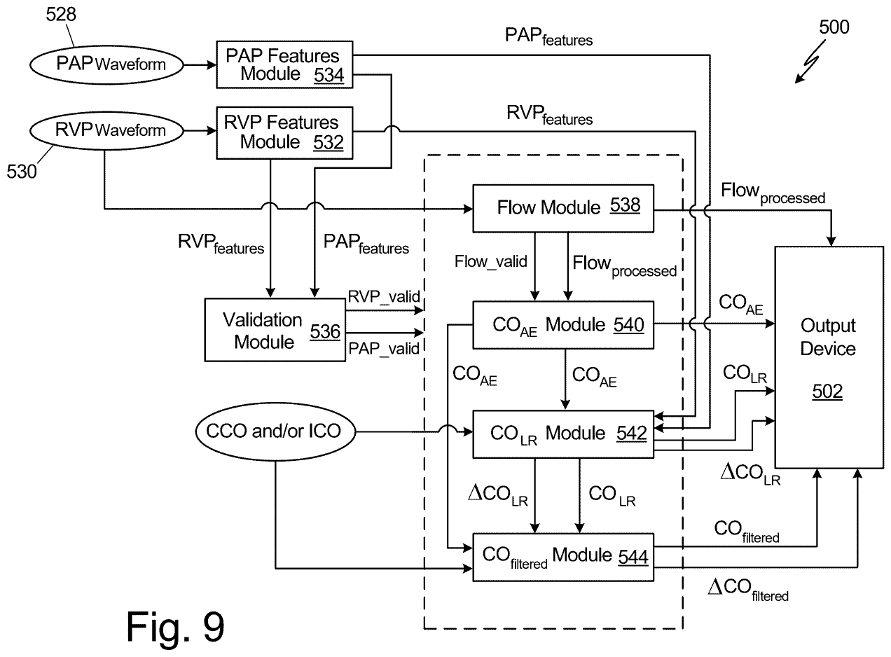

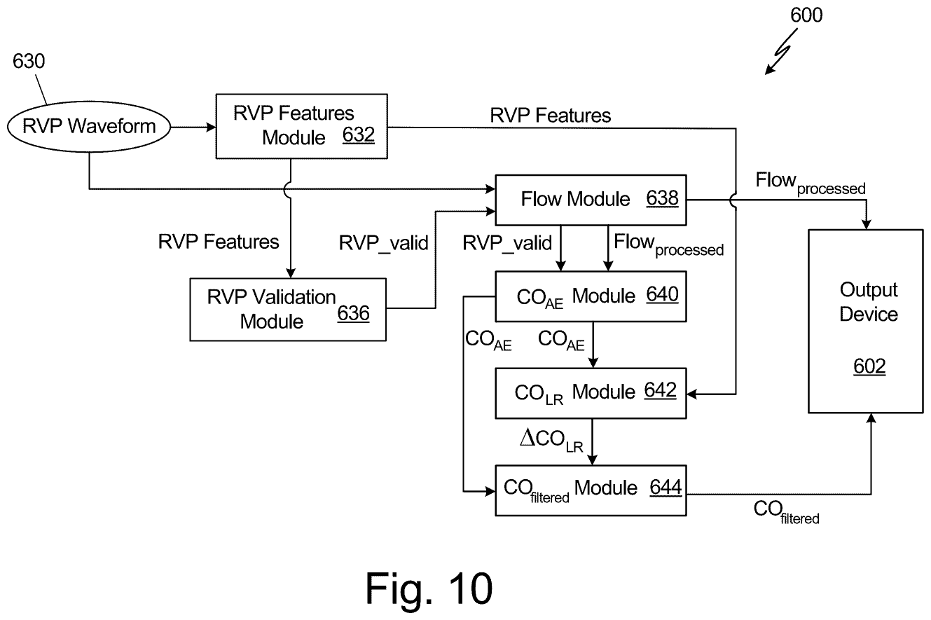

System memory 22 further includes RVCO module 38. RVCO module 38 includes one or more programs containing instructions to apply an RVCO algorithm to data received from catheter 54. The RVCO algorithm can be applied using the RVP waveform and the aforementioned RVP features, as derived from the RVP waveform. The RVCO algorithm can also be applied using the PAP waveform and aforementioned PAP features, as derived from the PAP waveform, in addition to the RVP features. Upon execution of RVCO module 38, the RVP waveform or the RVP and PAP waveforms are processed via the RVCO algorithm to produce one or more cardiac output parameters. In some examples, the one or more cardiac output parameters can include a continuous cardiac output. Various examples of the implementation of the RVCO algorithm are discussed in greater detail in the description of FIG. 9-10.

In some additional or alternative examples, the one or more cardiac output parameters are derived using a Swan-Ganz catheter. In other additional or alternative examples, system memory 22 includes instructions encoded in one or more programs directing system processor 20 to use an Arterial Pressure Cardiac Output (APCO) algorithm to derive the one or more cardiac output parameters. In such an example, the APCO algorithm can be used instead of the RVCO algorithm.

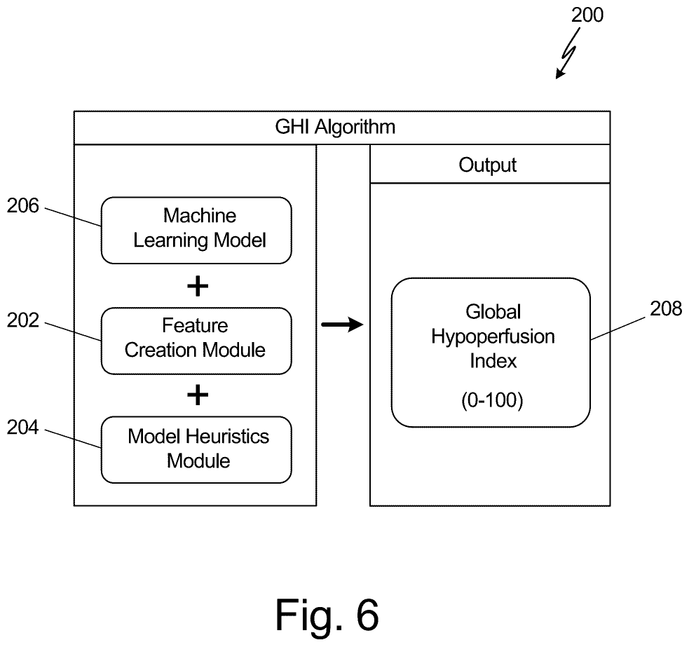

System memory 22 further includes GHI algorithm module 40. GHI algorithm module 40 includes one or more programs containing instructions to apply a GHI algorithm (i.e., GHI algorithm 200 of FIG. 6) to data generated by the execution of oximetry module 32, PAP features module 34, RVP features module 36, and RVCO module 38. Upon execution of GHI algorithm module 40, the SvO2 parameters, PAP features, RVP features, and cardiac output parameters are processed via the GHI algorithm. The GHI algorithm can include a feature creation model, model heuristics, and a machine learning model, wherein such models contribute to the creation of a global hypoperfusion index. The GHI algorithm will be described with respect to GHI algorithm 200 of FIG. 6.

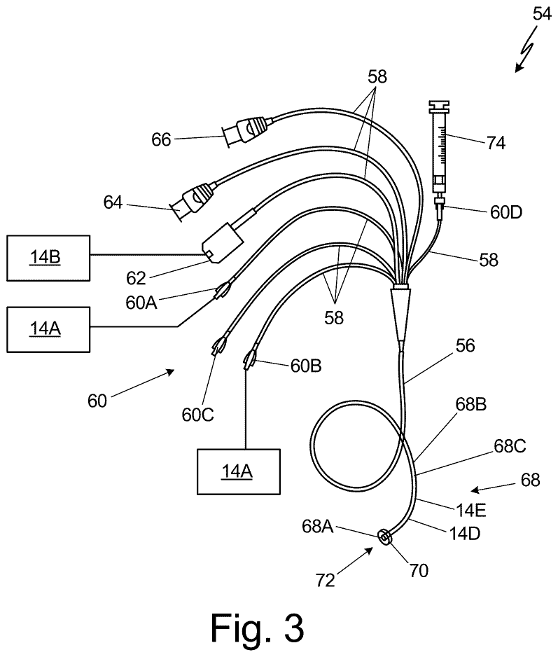

Hemodynamic monitor 12 is shown in FIG. 2. Catheter 54 is shown in FIG. 3. An example of a minimally invasive pressure sensor is shown in FIG. 4. An example of an oximetry module is shown in FIG. 5.

FIG. 2 is a perspective view of hemodynamic monitor 12. As illustrated in FIG. 2, hemodynamic monitor 12 includes display 24 that, in the example of FIG. 1, presents a graphical user interface 46 including control elements 48 (e.g., graphical control elements) that enable user interaction with hemodynamic monitor 12. Hemodynamic monitor 12 can also include a plurality of input and/or output (I/O) connectors 52 configured for wired connection (e.g., electrical and/or communicative connection) with one or more peripheral components, such as one or more hemodynamic sensors 14. While the example of FIG. 2 illustrates five separate I/O connectors 52, it should be understood that in other examples, hemodynamic monitor 12 can include fewer than five I/O connectors 52 or greater than five I/O connectors 52. In yet other examples, hemodynamic monitor 12 may not include I/O connectors 52, but rather may communicate wirelessly with various peripheral devices.

As described with respect to FIG. 1, hemodynamic monitor 12 includes one or more system processors 20 and system memory 22 that stores GHI software code 30, which is executable to determine a global hypoperfusion for patient 16. Hemodynamic monitor 12 can receive sensed hemodynamic data representative of oximetry data, an RVP waveform, and a PAP waveform, such as via one or more hemodynamic sensors 14 connected to hemodynamic monitor 12 via I/O connectors 52. Hemodynamic monitor 12 executes GHI software code 30 to determine a global hypoperfusion for patient 16, as is further described below.

As illustrated in FIG. 1, hemodynamic monitor 12 can present a graphical user interface at display 24. Display 24 can be an LCD, an LED display, an OLED display, or other display device suitable for providing information to users in graphical form. In some examples, such as the example of FIG. 2, display 24 can be a touch-sensitive and/or presence-sensitive display device configured to receive user input in the form of gestures, such as touch gestures, scroll gestures, zoom gestures, swipe gestures, or other gesture input.

Hemodynamic monitor 12 receives hemodynamic data from patient 16 via one or more hemodynamic sensors 14A, 14B, 14C, and 14D (collectively, hemodynamic sensors 14). In response to receiving hemodynamic data of patient 16, hemodynamic monitor 12 executes GHI software code 30 to determine a global hypoperfusion for patient 16 and display the global hypoperfusion information or other information on display 24. In some examples, hemodynamic monitor 12 can invoke a sensory alarm, such as an audible alarm, a haptic alarm, or other sensory alarm (for example, sensory alarm 50, as shown in FIG. 1) in response to determining that patient 16 is at risk of experiencing a global hypoperfusion event. Accordingly, hemodynamic monitor 12 can alert medical personnel of a potential global hypoperfusion event of patient 16.

FIG. 3 is a perspective view of catheter 54 that can be inserted into patient 16 and connected to one or more hemodynamic sensors 14 for providing hemodynamic data to hemodynamic monitor 12. For example, catheter 54 may be connected to one or more pressure-sensing hemodynamic sensors 14A for detecting RVP, PAP, or both right ventricular and pulmonary artery pressures of patient 16. Additionally, catheter 54 may interface with oximetry module 14B for sensing mixed venous oxygen saturation of patient 16. Protected by sheath 56, catheter 54 includes multiple lumens 58 that place fluid connectors 60, optical connector 62, thermistor connector 64, and thermal filament connector 66 in communication with one of ports 68, an embedded hemodynamic sensor 14C (e.g., a thermistor), or an embedded hemodynamic sensor 14D (e.g., a thermal filament). To facilitate insertion of catheter 54 within patient 16, or for certain hemodynamic measurements, catheter 54 includes balloon 70 located at tip 72 of catheter 54.

As shown in FIG. 3, catheter 54 includes distal port connector 60A communicating with port 68A at tip 72. Proximal injectate connector 60B communicates with proximal port 68B disposed approximately 30 cm from tip 72 and can be used for dispensing fluids and drugs into the patient's heart. Right ventricular pacing connector 60C communicates with right ventricle port 68C, which may be spaced approximately 19 cm from tip 72 or approximately 12 to 13 cm from tip 72. Connector 60C can be used for sensing RVP. Thermistor connector 64 electrically connects to hemodynamic sensor 14C (e.g., the thermistor) installed near tip 72 of catheter 54 for measuring core blood temperature within the pulmonary artery. In some examples of catheter 54, thermal filament connector 66 electrically connects to hemodynamic sensor 14D (e.g., the thermal filament) embedded within catheter 54 located within the patient's right ventricle. In some examples, catheter 54 does not include a thermal filament or corresponding thermal filament connector 66. Balloon connector 60D communicates with balloon 70 and with the use of syringe 74 can be used to inflate and deflate balloon 70.

After insertion into patient 16, e.g., via an introducer, distal port connector 60A and right ventricular pacing connector 60C can be connected to separate pressure transducer sensors 14A. A first pressure transducer 14A provides PAP waveform data sensed at distal port 68A located within the pulmonary artery of the patient's heart to hemodynamic monitor 12, while a second pressure transducer 14A provides RVP waveform data sensed at right ventricle port 68C located within the right ventricle of the patient's heart to hemodynamic monitor 12. Blood oxygen saturation data within the pulmonary artery can be provided by oximetry module 14B based on light pulses emitted from oximetry module 14B into the pulmonary artery and reflected light returns received by oximetry module 14B via optical connector 62 of catheter 54. Additionally, utilizing thermal filament connector 66 and thermistor connector 64 and associated cabling, hemodynamic monitor 12 can receive cardiac output data of patient 16 using, for example, a thermal dilution technique. The cardiac output measured via the thermal filament and corresponding thermal filament connector 66 can be considered a continuous cardiac output (labeled “CCO” in FIG. 1). If catheter 54 does not include a thermal filament, cardiac output can be determined using a thermistor after injecting a fluid bolus (or a set of boluses) of known volume and temperature via proximal injectate port 68B using the thermal dilution technique. The cardiac output measured via the thermistor and corresponding thermistor connector 64 after injection of the fluid bolus can be considered an intermittent cardiac output (labeled “ICO” in FIG. 1). Intermittent cardiac output measurements can be obtained at a frequency that is on the order of, e.g., minutes, hours, several hours, or even longer intervals, depending on the level of monitoring a patient requires. For example, a clinician may administer a bolus set of 3-4 fluid boluses, where one fluid bolus of the set is administered approximately every minute such that the complete bolus set lasts around three minutes. In one example, fluid boluses can be administered very frequently, such as every minute or every few minutes, when a clinician is assessing a patient's responsiveness to medication or another medical intervention. In another example, fluid boluses can be administered less frequently, such as every hour, every six hours, etc., if a patient is relatively stable in the ICU. Accordingly, catheter 54 can be used to provide a continuous cardiac output and/or an intermittent cardiac output.

Catheter 54 is one example of a catheter that can be used to measure SvO2, RVP, PAP, continuous cardiac output, and/or intermittent cardiac output. In other examples, any catheter configured to measure SvO2, RVP, PAP, continuous cardiac output and/or intermittent cardiac output can be used. For example, any right heart/pulmonary catheter, for example a Swan Ganz catheter, can be used.

FIG. 4 is a perspective view of hemodynamic sensor 14A that can be attached to patient 16 for sensing hemodynamic data representative of RVP or PAP of patient 16. As illustrated in FIG. 4, hemodynamic sensor 14A includes housing 76, fluid input port 78, catheter-side fluid port 80, and I/O cable 82. Fluid input port 78 is configured to be connected via tubing or other hydraulic connection to a fluid source, such as a saline bag or other fluid input source. Catheter-side fluid port 80 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). The catheter can also be inserted within a ventricular system of the patient. I/O cable 82 is configured to connect to hemodynamic monitor 12 via, e.g., one or more of I/O connectors 52 (shown in FIG. 2). Housing 76 of hemodynamic sensor 14A encloses one or more pressure transducers, communication circuitry, processing circuity, and corresponding electronic components to sense fluid pressure corresponding to the RVP or PAP of patient 16 that is transmitted to hemodynamic monitor 12 (shown in FIG. 2) via I/O cable 82.

In operation, a column of fluid (e.g., saline solution) is introduced from a fluid source (e.g., a saline bag) through hemodynamic sensor 14A via fluid input port 78 to catheter-side fluid port 80 toward the catheter inserted into an arterial system and/or a ventricular system of patient 16. RVP or PAP is communicated through the fluid column to pressure sensors located within housing 76 which sense the pressure of the fluid column. Hemodynamic sensor 14A translates the sensed pressure of the fluid column to an electrical signal via the pressure transducers and outputs the corresponding electrical signal to hemodynamic monitor 12 (shown in FIG. 1) via I/O cable 82. Hemodynamic sensor 14 therefore transmits analog sensor data (or a digital representation of the analog sensor data) to hemodynamic monitor 12 (shown in FIG. 1) that is representative of substantially continuous beat-to-beat monitoring of the RVP or PAP of patient 16.

FIG. 5 is a perspective view of oximetry module 14B for receiving oximetry data from a catheter inserted within patient 16. As depicted in FIG. 5, hemodynamic sensor 14B includes an optical transmitter and an optical receiver arranged to electrically communicate to a catheter via I/O connector 84 installed within housing 86 and accessible via protective door 88. Within housing 86, hemodynamic sensor 14B, as depicted by FIG. 5, includes communication circuitry, processing circuity, and corresponding electronic components to sense blood oxygen saturation data derived from optical light emissions transmitted via a catheter into a patient and corresponding light returns received from patient 16 via the catheter. An electrical signal indicative of the patient blood oxygen saturation levels is transmitted to hemodynamic monitor 12 via cable 90 and connector 92, which interfaces with one of I/O connectors 52 (shown in FIG. 2).

FIG. 6 is a block diagram of GHI algorithm 200. GHI algorithm 200 includes feature creation module 202, model heuristics module 204, and machine learning model 206. The output of GHI algorithm 200 is global hypoperfusion index 208. GHI algorithm 200 is one example of GHI algorithm module 40 shown in FIG. 1.

The feature creation module 202 within GHI algorithm 200 can be a model to compute one or more intermediate features based upon the RVP features, the cardiac output parameters, the PAP features, and the SvO2 parameters. Such intermediate features can include, for example, an arterial elastance, calculated by dividing the mean PAP (i.e., from the PAP features) by the stroke volume (i.e., from the cardiac output parameters). Such intermediate features can also include a pulmonary vascular resistance, calculated by dividing the mean PAP (i.e., from the PAP features) by the continuous cardiac output (i.e., from the cardiac output parameters). The listed intermediate features are merely intended to be examples, and a plurality of additional or alternative features can be calculated by feature creation module 202 within GHI algorithm 200.

Model heuristics module 204 within GHI algorithm 200 can function as validation checks on the RVP features, the cardiac output parameters, the PAP features, and the SvO2 parameters. Such validation checks can be performed to determine whether the data is valid for use within GHI algorithm 200. Data that shows significant abnormalities (e.g., SvO2 parameters with a low SQI) may be discarded such that it is not used in the calculation of global hypoperfusion index 208. A similar SQI can be generated for the PAP and the RVP waveforms to determine the integrity of the corresponding data. Other methods of evaluating data integrity can also be used within model heuristics module 204 to ensure that GHI algorithm 200 is not premised on faulty data.

Machine learning model 206 within GHI algorithm 200 can be a predictive risk model that is based upon a linear, weighted set of predictive features that have been identified as being predictive of a global hypoperfusion event. In some examples, the features predictive of global hypoperfusion are identified using a regression model that minimizes loss. Predictive features can include the SvO2 parameters, PAP features, RVP features, and cardiac output parameters are processed via GHI algorithm 200. In some examples, GHI algorithm 200 is configured to predict when the SvO2 of a patient will drop below 60%, indicating a global hypoperfusion event. Such a prediction may be made, for example, thirty minutes to one hour prior to the occurrence of a global hypoperfusion event. The predictive timing can be greater or less than the indicated range in other examples. The machine learning model can be a deep-learning model that uses neural network architecture.

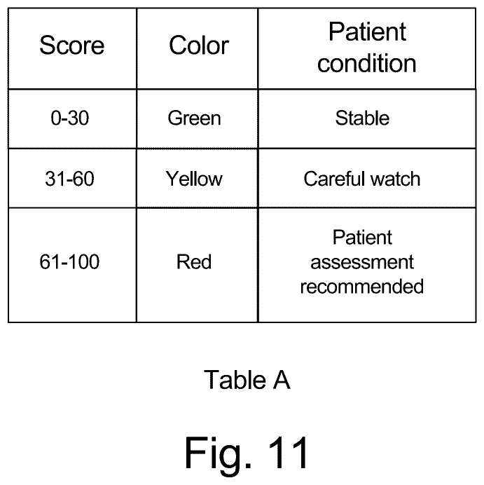

Global hypoperfusion index 208 generated by GHI algorithm 200 can be a numerical value on a defined scale, wherein the numerical value corresponds to the likelihood of a global hypoperfusion event occurring. In some examples, the numerical values are separated into ranges, wherein each range indicates a risk level based on determined thresholds. For example, global hypoperfusion index 208 can be a numerical score on a scale of 0-100. A score of 0 -30 may indicate a stable patient condition, a score of 31-60 may indicate a patient that should be watched for global hypoperfusion, and a score of 61-100 may indicate that patient assessment is recommended. Global hypoperfusion index 208 and the various methods of implementation with numerical thresholds are discussed in greater detail in the description of FIG. 11.

Upon determining global hypoperfusion index 208 and corresponding patient risk level, such information is transmitted from processor 20 to display 24 via digital-to-analog converter 28 (shown in FIG. 1). Display 24 can, for example, be a patient monitor which is viewable by a clinician. Display 24 can additionally or alternatively be a liquid crystal display (LCD), a light-emitting diode (LED) display, an organic light-emitting diode (OLED) display, or other display device suitable for providing information to users. Processor 20 can also be configured to trigger a sensory alarm via display 24 if the global hypoperfusion index indicates that patient assessment is recommended.

Hemodynamic monitoring system 10 and GHI algorithm 200 (one example of GHI algorithm module 40 that can be used in GHI software code 30) provide various advantages. System 10 allows for predicting a global hypoperfusion event of a patient based on parameters received from catheter 54. Further, system 10 does so with a high degree of precision and does so sufficiently in advance to allow for action to be taken by clinicians. System 10 also provides the advantage of continuously updating the global hypoperfusion index on display 24, thereby allowing the clinician or other viewer to determine real-time health data of the patient. The sensory alarm and display 24 allow for a clinician to be notified of a potential future global hypoperfusion event and respond accordingly. In some examples, display 24 can be used to display a graph of the change in the venous oxygen saturation value over time.

FIG. 7 depicts a graph illustrating right ventricular pressure (RVP) waveform trace 300 including example indicia indicative of blood flow, cardiac output, and global hypoperfusion. RVP waveform trace 300 includes indicia 306, 308, 310, 312, 314, 316, 318, and 320. RVP waveform trace 300 corresponds to hemodynamic data sensed by catheter 54 and more specifically by hemodynamic sensor 14A (shown in FIG. 1). RVP waveform trace 300 (represented via digital hemodynamic data) can include various indicia indicative of blood flow, cardiac output, and global hypoperfusion for patient 16. The right ventricular features are extracted from RVP waveform trace 300 (i.e., the RVP waveform) via execution of RVP features module 36 (shown in FIG. 1). In some examples, prior to extracting indicia from RVP waveform grace 300, a beat detector algorithm identifies the start and end of individual heartbeats for the RVP waveform. Beat detection algorithms can identify the start of the heartbeat based on the maximum RVP, the minimum RVP, the maximum or minimum rate of change in RVP, and/or the second derivative with respect to time in the RVP. After heartbeat identification within the RVP waveform, various indicia of blood flow, cardiac output, and global hypoperfusion can be extracted from the RVP waveform on an on-going, beat-to-beat basis.

Indicium 306 of RVP waveform trace 300 corresponds to the minimum diastolic pressure. Indicium 308 of RVP waveform trace 300 corresponds to the end diastolic pressure. Indicium 310 of RVP waveform trace 300 corresponds to the maximum systolic pressure. Indicium 312 of RVP waveform trace 300 corresponds to the end systolic pressure. Slope S1 is the slope of RVP waveform trace 300, which may also provide indicia. Slope S1 is depicted at one location but is representative of multiple slopes that may be determined at multiple locations along RVP waveform trace 300. For instance, indicium 314, corresponding to the maximum pressure rate of change with respect to time (dP/dt) during systolic rise, and indicium 316, corresponding to the minimum pressure rate of change with respect to time (dP/dt) during the relaxation period after end systole, are further example indicia. Similarly, the second time derivative of RVP waveform trace 300 may be determined at any location along the waveform and used as indicia. Other exemplary indicia include RVP gradients, or the difference in pressure at different times during diastolic or systolic phase. For example, indicium 318 corresponds to the diastolic gradient, or the difference between minimum diastolic pressure (indicium 306) and end diastolic pressure (indicium 308). Indicium 320 corresponds to the right ventricular pulse pressure, or a pressure gradient equal to the difference between end diastolic pressure (indicium 308) and maximum systolic pressure (indicium 310).

Additional indicia can be extracted from RVP waveform trace 300 during various intervals by execution of RVP features module 36 stored in system memory 22 (shown in FIG. 1). For instance, systolic rise (indicia 308-310), systolic decay (310-312), isovolumetric relaxation (indicia 312-306), diastolic phase (indicia 306-308), and the heartbeat interval (between indicia 306) can be determined by execution of RVP features module 36. Such indicia may include the mean RVP during one or more time intervals.

FIG. 8 depicts a graph illustrating pulmonary artery pressure (PAP) waveform trace 400 including example indicia indicative of blood flow, cardiac output, and global hypoperfusion. PAP waveform trace 400 includes indicia 424, 426, 428, 430, and 432. PAP waveform trace 400 corresponds to hemodynamic data sensed by catheter 54 and more specifically, by hemodynamic sensor 14A (shown in FIG. 1). PAP waveform trace 400 (represented via digital hemodynamic data) can include various indicia indicative of blood flow, cardiac output, and global hypoperfusion for patient 16. The PAP features are extracted from PAP waveform trace 400 (i.e., the PAP waveform) via execution of PAP features module 34 (shown in FIG. 1). In some examples, prior to extracting indicia from PAP waveform trace 400, a beat detector algorithm identifies the start and end of individual heartbeats for the PAP waveform. Beat detection algorithms can identify the start of the heartbeat based on the maximum PAP, the minimum PAP, the maximum or minimum rate of change in PAP, and/or the second derivative with respect to time in the PAP. After heartbeat identification within the PAP waveform, various indicia of blood flow, cardiac output, and global hypoperfusion can be extracted from the PAP waveform on an on-going, beat-to-beat basis.

Indicium 424 of PAP waveform trace 400 corresponds to the start of a heartbeat. Indicium 426 of PAP waveform trace 400 corresponds to the maximum systolic pressure marking the end of systolic rise. Indicium 428 of PAP waveform trace 400 corresponds to the presence and pressure of the dicrotic notch marking the end of systolic decay. Indicium 430 of PAP waveform trace 400 corresponds to the minimum diastolic pressure of the heartbeat of patient 16. The mean PAP can also be an indicium. PAP gradients, or pressure differences between points of the PAP waveform trace 400, can be indicia. For instance, indicium 432 corresponds to pulmonary pulse pressure, or the difference between minimum diastolic pressure (indicium 430) and maximum systolic pressure (indicium 426). S2 is the slope of PAP waveform trace 400, which may also provide indicia. Slope S2 is depicted at one location but is representative of multiple slopes that may be determined at multiple locations along PAP waveform trace 400. For instance, indicia may include the maximum and/or minimum time derivative of PAP waveform trace 400.

Additional indicia can be extracted from PAP waveform trace 400 during various intervals by execution of PAP features module 34 of system memory 22 (shown in FIG. 1). For instance, the interval from the maximum systolic pressure at indicium 426 to the diastole at indicium 428 and the interval from the start of the heartbeat at indicium 424 to the diastole at indicium 430 can be extracted from PAP waveform trace 400. Execution of PAP features module 34 may identify additional indicia from PAP waveform trace 400 during various intervals. For instance, systolic rise (indicia 424-426), systolic decay (indicia 426-428), systolic phase (indicia 424-428), diastolic phase (indicia 428-430), and the heartbeat interval (between indicia 424) can be determined by execution of PAP features module 34. Such indicia may include the mean PAP during one or more time intervals. The area under the curve of PAP waveform trace 400 and the standard deviations of PAP waveform trace 400 determined for the above-referenced intervals can also serve as additional indicia for patient 16.

FIG. 9 is a diagram of an example of system 500 for implementing a right ventricular cardiac output (RVCO) algorithm. System 500 can be included within system 100 of FIG. 1 within, for example, system memory 22. System 500 is described as a standalone system herewith, but components of system 500 can also be shared with components of system 100 shown in FIG. 1. System 500 can be used in connection with any suitable catheter, for example catheter 54 as shown in FIG. 1. System 500 includes RVP features module 532, PAP features module 534, validation module 536, flow module 538, CO_{AE} module 540 (a module that estimates cardiac output based on an autoencoder model), CO_{LR} module 542 (a module that estimates cardiac output using a linear regression model), CO_{filtered} module 544 (a module that filters one or more estimates of cardiac output), and output device 102. RVP features module 532 can be RVP features module 36 and PAP features module 534 can be PAP features module 34 of system 100 shown in FIG. 1, respectively, but are described as part of system 500 herewith for clarity. Each of validation module 536, flow module 538, CO_{AE} module 540, CO_{LR} module 542, and CO_{filtered} module 544 can be included in RVCO module 38 of system 100 shown in FIG. 1. The number and organization of modules within system 100 can be greater or fewer than those described herein as a result of software code organization. The modules of system 500 will be described sequentially; however, these modules can also include overlapping or interspersed functionality.

RVP features module 532 includes methods in code for extracting RVP features from RVP waveform 530. RVP waveform 530 is akin to RVF waveform trace 300 of FIG. 7. RVP features module 532 receives RVP waveform 530 as an input. RVP waveform 530 corresponds to hemodynamic data sensed by catheter 54, and more specifically by hemodynamic sensor 14A. The sensed hemodynamic data to which RVP waveform 530 corresponds is passed to RVP features module 532. RVP features module 532 extracts RVP features from RVP waveform 530. RVP features module 532 outputs RVP features to validation module 536. RVP features module 532 also outputs RVP features to CO_{LR} module 542.

PAP features module 534 includes methods in code for extracting PAP features from PAP waveform 528. PAP waveform 528 is akin to PAP waveform trace 400 of FIG. 8. PAP features module 534 receives PAP waveform 528 as an input. PAP waveform 528 corresponds to hemodynamic data sensed by catheter 54, and more specifically by hemodynamic sensor 14A. The sensed hemodynamic data to which PAP waveform 528 corresponds is passed to PAP features module 534. PAP features module 534 extracts PAP features from PAP waveform 528. PAP features module 534 outputs PAP features to validation module 536. PAP features module 534 also outputs RVP features to CO_{LR} module 542.

Validation module 536 includes methods in code for cleaning data and ensuring RVP waveform 530 and PAP waveform 528 are valid and reliable. Validation module 536 receives RVP waveform 530, PAP waveform 528, RVP features, and PAP features as inputs. Validation module 536 can analyze RVP waveform 530 and/or PAP waveform 528 in ten-second segments. A ten-second segment is merely one example, and it is understood that validation module can use any other suitable time window to evaluate RVP waveform 530 and PAP waveform 528. Within the applicable time window, a signal quality detector within validation module 536 can analyze RVP waveform 530 and PAP waveform 528 and assign a signal quality index (SQI) to each waveform based upon a signal quality algorithm. The signal quality algorithm can analyze data by comparing RVP waveform 530 to PAP waveform 528 and/or by identifying artifacts in RVP waveform 530 to PAP waveform 528 such as negative pressure, inordinately high pressure, or other data that is not physiologically possible. Values outside of the physiologically possible data ranges can be discarded, and thus RVP waveform 530 and PAP waveform 528 can be smoothed by such a process.

Validation module 536 outputs instructions indicating whether valid RVP (“RVP_valid”) and valid PAP (“PAP_valid”) are true or false to the modules contained in the dashed line box shown in FIG. 9 (i.e., flow module 538, CO_{AE} module 540, CO_{LR} module 542, and CO_{filtered} module 544) and can output the determination indicating whether RVP waveform 530 and PAP waveform 528 are valid to output device 502, such as display 24 (shown in FIG. 1). A determination of validity can be made based upon the SQI for each waveform derived from the signal quality algorithm. An instruction that RVP_valid and PAP_valid are true means that hemodynamic data corresponding to RVP waveform 530 and PAP waveform 528 is valid for further processing by the modules contained in the dashed line box. An instruction that RVP_valid and PAP_valid are false means that hemodynamic data corresponding to RVP waveform 530 and PAP waveform 528 is not valid for further processing. Validation module 536 also outputs a smoothed RVP waveform to flow module 538 and outputs RVP features and PAP features to CO_{LR} module 542.

Flow module 538 includes methods in code for estimating a blood flow waveform from RVP waveform 530. Flow module 538 receives RVP waveform 530 as an input. Flow module 538 can also receive the smoothed RVP waveform from validation module 536. RVP waveform 530 can be transformed and processed via an autoencoder model and a filter. The autoencoder model can result in an estimated waveform of raw blood flow, and the filter can further process the estimated waveform based on physiological expectations (e.g., removing negative flow, excessively high flow values, inordinately dissimilar flow per beat, etc.) to generate flow_{processed}. The filter can also generate an SQI corresponding to flow_{processed}. Based on the SQI, flow module 538 can generate an indication of whether or not flow_{processed} is valid.

Flow module 538 outputs instructions indicating whether valid flow (“flow_valid”) is true or false to CO_{AE} module 540 and can output the determination indicating whether processed blood flow (“flow_{processed}”) is valid to output device 502, such as display 24 (shown in FIG. 1). An instruction that flow_valid is true means that the estimated blood flow waveform, flow_{processed}, is valid for further processing by CO_{AE} module 540. An instruction that flow_valid is false means that the estimated blood flow waveform, flow_{processed}, is not valid for further processing. Flow module 538 further outputs flow_{processed} to CO_{AE} module 540. Flow module 538 can also output flow_{processed} to output device 502.

CO_{AE} module 540 includes methods in code for deriving cardiac output from the estimated blood flow waveform. CO_{AE} module 540 receives flow_{processed} as an input. Flow_{processed} is fed to CO_{AE} module 540 from flow module 538. CO_{AE} module 540 also receives an instruction from flow module 538 indicating whether flow_valid is true or false such that CO_{AE} module 540 can proceed or not proceed accordingly. If instructed to proceed, CO_{AE} module 540 calculates an average value of the flow_{processed} waveform to generate an estimate of cardiac output. The estimate can be calculated, for example, for a ten-second portion of flow_{processed}. CO_{AE} module 540 outputs autoencoder cardiac output (“CO_{AE}”) to CO_{LR} module 542 and CO_{filtered} module 544. CO_{AE} module 540 can also output CO_{AE} to output device 502.

CO_{LR} module 542 includes methods in code for estimating change in cardiac output and cardiac output based on RVP features and PAP features. CO_{LR} module 542 receives RVP features, PAP features, CO_{AE}, CCO, and iCO as inputs. RVP features are fed to CO_{LR} module 542 from RVP features module 532 or validation module 536. Likewise, PAP features are fed to CO_{LR} module 542 from PAP features module 534 or validation module 536. CO_{AE} is fed to CO_{LR} module 542 from CO_{AE} module 540. CCO and iCO correspond to cardiac output data of the patient received from catheter 54. CCO and iCO are passed to CO_{LR} module 542.

CO_{LR} module 542 can include a regression model configured to estimate the change in cardiac output over time for the patient. The regression model can be a machine learning model trained with a training data set wherein change in cardiac output and corresponding changes in RVP features and PAP features are known for a patient. Thus, when provided with RVP features and PAP features for a determined time window, the regression model can determine a linear regression change in cardiac output (“ΔCO_{LR}”) based on a set of reference RVP features and PAP features that correspond to a reference time window. In some examples, demographic information and SvO2 are also fed to the regression model in addition to the RVP features and PAP features.

CO_{LR} module 542 can also include a cardiac output estimator which then calculates a linear regression cardiac output (“CO_{LR}”) based upon ΔCO_{LR}. In some examples, CO_{AE}, CCO and iCO are also fed to the cardiac output estimator. In such an example, the value of CO_{AE}, the measurement of CCO, and the measurement of iCO corresponding to the reference time window and are used as a starting point from which to compute CO_{LR}. CO_{LR} module 542 outputs linear regression cardiac output (“CO_{LR}”) and linear regression change in cardiac output (“ΔCO_{LR}”) to CO_{filtered} module 544. CO_{LR} module 542 can also output CO_{LR} and ΔCO_{LR} to output device 502.

CO_{filtered} module 544 includes methods in code for filtering cardiac output or change in cardiac output estimates via, for example, a Kalman filter algorithm. CO_{filtered} module 544 receives CO_{AE}, CO_{LR}, CCO, iCO, and ΔCO_{LR} as inputs. CO_{AE} is fed to CO_{filtered} module 544 from CO_{AE} module 540. CO_{LR} and ΔCO_{LR} are fed to CO_{filtered} module 544 from CO_{LR} module 542. In the same manner as described above for CO_{LR} module 542, CCO and iCO are also passed to CO_{filtered} module 544. CO_{filtered} module 44 outputs filtered cardiac output (“CO_{filtered}”) and filtered change in cardiac output (“ΔCO_{filtered}”) to output device 102.

Output device 502 is a device for receiving outputs from system 500. Output device 502 can include display 24, as shown in FIG. 1. For example, output device 502 can receive final estimates of flow, cardiac output, and/or change in cardiac output for display via display 24. Output device 502 can receive outputs from flow module 538, CO_{AE} module 540, CO_{LR} module 542, and CO_{filtered} module 544. More specifically, output device 502 receives flow_{processed}, CO_{AE}, CO_{LR}, ΔCO_{LR}, CO_{filtered}, and ΔCO_{filtered}. Each of these outputs can be displayed via display 24 as corresponding graphs representing the values over time.

Although several modules are illustrated in FIG. 9 as having multiple inputs and outputs, some of the inputs and outputs are optional, and many configurations of system 500 are possible. In some examples, PAP waveform, CCO, and/or iCO may not be used. In some examples, an estimate of cardiac output for the patient can be output from any one or more of CO_{AE} module 540, CO_{LR} module 542, and CO_{filtered} module 544. These configurations can depend, for example, on the combination of hemodynamic sensors within catheter 52 (shown in FIG. 1) used or the desired output. FIG. 10 depicts one such alternative, wherein the cardiac output is calculated via a different set of inputs than the example depicted in FIG. 9.

FIG. 10 is a diagram of system 600, which is an alternative example of system 500 for implementing an RVCO algorithm. System 600 can be included within system 100 of FIG. 1 within, for example, system memory 22. System 600 is described as a standalone system herewith, but components of system 600 can also be shared with components of system 100 shown in FIG. 1. System 600 can be used in connection with any suitable catheter, for example catheter 54 as shown in FIG. 1. System 600 includes RVP waveform 630, RVP features module 632, validation module 636, flow module 638, CO_{AE} module 640, CO_{LR} module 642, CO_{filtered} module 644, and output device 602. RVP features module 632 can be RVP features module 36 of system 100 shown in FIG. 1, but is described as part of system 600 herewith for clarity. Each of RVP waveform 630, RVP features module 632, validation module 636, flow module 638, CO_{AE} module 640, CO_{LR} module 642, CO_{filtered} module 644, and output device 602 is generally similar to the component or module with the same name and reference number decremented by 100 as described above with reference to FIG. 9, and one possible configuration of these components and modules is described here.

As shown in FIG. 10, RVP features module 632 receives RVP waveform 620 as an input. RVP features module 632 extracts RVP features from RVP waveform 630. RVP features module 632 outputs RVP features to validation module 636. RVP features module 632 also outputs RVP features to CO_{LR} module 642.

Validation module 636 receives RVP waveform 630 and RVP features as inputs. RVP features are fed to validation module 636 from RVP features module 632. Validation module 636 outputs instructions indicating if RVP_valid is true or false to flow module 638. An instruction that RVP_valid is true means that hemodynamic data corresponding to RVP waveform 630 is valid for further processing by flow module 638. An instruction that RVP_valid is false means that hemodynamic data corresponding to RVP waveform 630 is not valid for further processing. Validation module 636 also outputs a smoothed RVP waveform to flow module 638.

Flow module 638 receives the smoothed RVP waveform from validation module 636 as an input. Flow module 638 outputs instructions indicating whether flow_valid is true or false to CO_{AE} module 640. An instruction that flow_valid is true means that the estimated blood flow waveform, flow_{processed}, is valid for further processing by CO_{AE} module 640. An instruction that flow_valid is false means that the estimated blood flow waveform, flow_{processed}, is not valid for further processing. Flow module 638 further outputs flow_{processed} to CO_{AE} module 640. Flow module 638 also outputs flow_{processed} to output device 602.

CO_{AE} module 640 receives flow_{processed} as an input. Flow_{processed} is fed to CO_{AE} module 640 from flow module 638. CO_{AE} module 640 also receives an instruction from flow module 638 indicating whether flow_valid is true or false such that CO_{AE} module 640 can proceed or not proceed accordingly. CO_{AE} module 640 outputs CO_{AE} to CO_{LR} module 642 and CO_{filtered} module 644.

CO_{LR} module 642 receives RVP features and CO_{AE} as inputs. RVP features are fed to CO_{LR} module 642 from RVP features module 632. CO_{AE} is fed to CO_{LR} module 642 from CO_{AE} module 640. CO_{LR} module 642 outputs CO_{LR} and ΔCO_{LR} to CO_{filtered} module 644.

CO_{filtered} module 644 receives CO_{AE}, CO_{LR}, and ΔCO_{LR} as inputs. CO_{AE} is fed to CO_{filtered} module 644 from CO_{AE} module 640. CO_{LR} and ΔCO_{LR} are fed to CO_{filtered} module 644 from CO_{LR} module 642. CO_{filtered} module 644 outputs CO_{filtered} to output device 602.

As shown in FIG. 6, output device 602 receives outputs from flow module 638 and CO_{filtered} module 644. More specifically, output device 602 receives flow_{processed} and CO_{filtered}. Each of these outputs can be displayed (e.g., via display 24, as shown in FIG. 1) as corresponding graphs representing the values over time.

Systems 500 and 600 are systems implementing a right ventricular cardiac output (RVCO) algorithm. Implementing such an algorithm in the context of system 100 for generating a GHI allows for a reliable cardiac output to be used within GHI algorithm 200. Further, systems 500 and 600 also generate intermediate data which can be displayed via display 24. Such data may inform a clinician of key parameters such as flow_{processed} and CO_{filtered} in addition to the GHI index. Thus, systems 500 and 600 are advantageous for the parameters provided in the calculation of the GHI and for the output of other key parameters for clinician use.

FIG. 11 is a table showing an example of an output of system 100 for generating the GHI. FIG. 11 includes Table A, wherein Table A contains various threshold values indicative of the risk level of a patient condition based upon the derived GHI. In the example of Table A, a GHI of 0 -30 is indicative of a stable patient condition. A GHI of 31-60 is indicative of a “careful watch” condition, wherein a clinician is recommended to watch the patient in case the condition worsens. A GHI of 61-100 indicates that patient assessment is recommended, and the clinician should take further action. In such an example, a GHI of 61-100 can indicate that a predicted venous oxygen saturation value (SvO2) will go below 60.

Table A is merely one example of an implementation of an output of system 100. In other examples, the threshold values for the GHI corresponding to a given risk level can be adjusted. In still other examples, there may be two, three, or more than three different risk levels with predetermined GHI ranges corresponding to each risk level.

In some examples, the output of system 100 can include a sensory alarm. Thus, in the example of Table A, a sensory alarm can be output once the patient reaches a GHI of 61-100, indicating that patient assessment is recommended. In other examples, various sensory alarms can be used in order to indicate different risk levels (e.g., a different sensory alarm for a “careful watch” condition versus a “patient assessment recommended” condition). In some examples, display 24 can be used to display a graph of the change in the venous oxygen saturation value over time.

In some examples, display 24 can display, in addition to the GHI and sensory alarm, secondary screening information. The secondary screening information can be indicative of one or more possible causes of the patient condition when the risk level indicates that a patient assessment is required. The secondary screening information can be information indicative of whether cardiac output is related to the patient condition or whether oxygen uptake is related to the patient condition. In the case where neither cardiac output nor oxygen uptake are at issue, secondary screening information may be omitted.

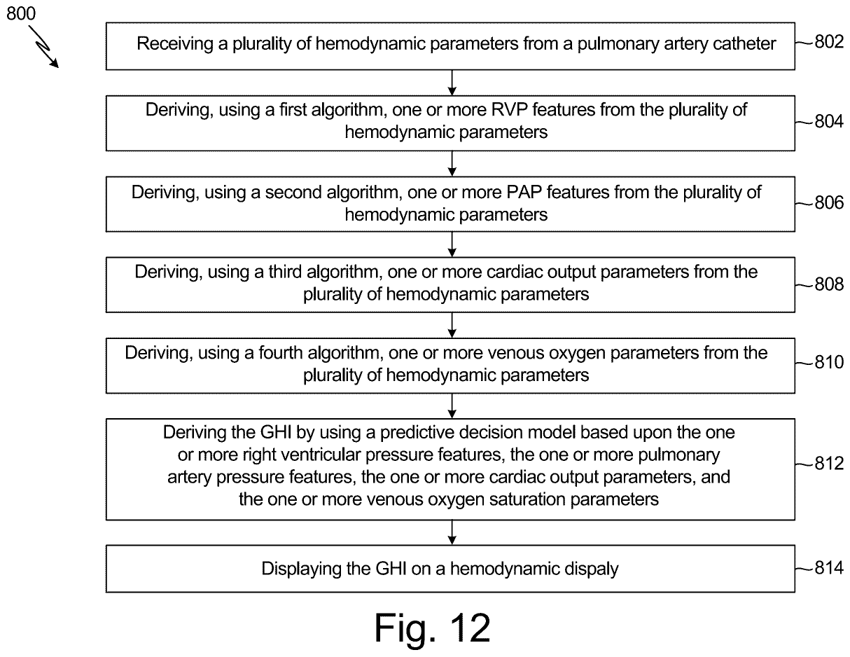

FIG. 12 is a method flow diagram illustrating method 800 for generating the GHI. Method 800 will be described with respect to the reference numerals of system 100 for clarity. Method 800 includes steps 802-814.

Method 800 begins at step 802, wherein hemodynamic monitor 12 receives a plurality of hemodynamic parameters from catheter 54. Catheter 54 includes a plurality of hemodynamic sensors, such as hemodynamic sensors 14A, 14B, 14C, and 14D, configured to output an SvO2 value, an RVP waveform, and a PAP waveform.

At step 804, system processor 20 of hemodynamic monitor 12 derives, using a first algorithm, one or more RVP features from the plurality of hemodynamic parameters. The first algorithm can be executed upon execution of RVP features module 36 by processor 20. The first algorithm can be executed upon the RVP waveform, as received from catheter 54.

At step 806, system processor 20 of hemodynamic monitor 12 derives, using a second algorithm, one or more PAP features from the plurality of hemodynamic parameters. The second algorithm can be executed upon execution of PAP features module 34 by processor 20. The second algorithm can be executed upon the PAP waveform as received from catheter 54.

At step 808, system processor 20 of hemodynamic monitor 12 derives, using a second algorithm, one or more cardiac output parameters from the plurality of hemodynamic parameters. The second algorithm can be executed upon execution of RVCO module 38 by processor 20. The second algorithm can be executed upon the RVP waveform, PAP waveform, and/or SvO2 data (or any combination thereof) as received from catheter 54.

At step 810, processor 20 of hemodynamic monitor 12 derives, using a fourth algorithm, one or more SvO2 parameters from the plurality of hemodynamic parameters. The fourth algorithm can be executed upon execution of oximetry module 32 by processor 20. The fourth algorithm can be executed upon the SvO2 parameters as received from catheter 54.

Steps 804, 806, 808, and 810 can be completed in any order. Further, any of steps 804, 806, 808, and 810 can be completed at the same time or at different times.

At step 812, processor 20 of hemodynamic monitor 12 derives the GHI. Processor 20 derives the GHI by using a predictive decision model based upon the one or more RVP features, the one or more PAP features, the one or more cardiac output parameters, and the one or more venous oxygen saturation parameters. The derivation of the GHI can be done upon execution of GHI algorithm module 40 by processor 20.

At step 814, hemodynamic monitor 12 transmits the GHI to display 24, where the GHI is displayed. The GHI can be displayed along with a risk level based on, for example, one or more predetermined risk level thresholds, such as those described with respect to Table A of FIG. 11.

The techniques of this disclosure describe a system for determining a global hypoperfusion index (GHI). The GHI is used to predict a future global hypoperfusion event in a patient. The system is advantageous as it uses a variety of hemodynamic parameters combined with a predictive machine learning model in order to accurately determine the likelihood of a future global hypoperfusion event. Because of this, clinicians can be forewarned of such an event and take action before such an event may occur. The system further generates a variety of useful hemodynamic parameters that are then viewable by the clinician on a display device. The system includes outputs that are clinician friendly and easy to interpret. The outputs are in line with clinical hemodynamic parameter thresholds used to identify hemodynamic unstable patients.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

A system for determining a global hypoperfusion index (GHI) representative of a prediction of a future global hypoperfusion event within a patient includes an arterial blood pressure sensor, a ventricular blood pressure sensor, an oximetry module, and an integrated hardware unit. The arterial blood pressure sensor includes a first housing, a first fluid input port connected via tubing to a first fluid source, a first catheter-side fluid port connected to a catheter inserted within an arterial system of a patient, first pressure transducer in communication with the first fluid source through the first catheter-side fluid port, and a first I/O cable in electrical communication with the first pressure transducer. The ventricular blood pressure sensor includes a second housing, a second fluid input port connected via tubing to a second fluid source, a second catheter-side fluid port connected to the catheter inserted within a ventricular system of a patient, second pressure transducer in communication with the second fluid source through the second catheter-side fluid port, and a second I/O cable in electrical communication with the second pressure transducer. The oximetry module includes an optical transmitter, an optical receiver, and an I/O cable in electrical communication with the optical transmitter and the optical receiver. The integrated hardware unit includes a system processor, a system memory, a display including a user interface, and an analog-to-digital (ADC) converter. The system memory includes instructions that, when executed by the system processor, cause the system to perform the following steps: receive arterial hemodynamic data from the arterial blood pressure sensor; receive ventricular hemodynamic data from the ventricular blood pressure sensor; receive blood oxygen saturation data from the oximetry module; derive, using a first algorithm, one or more right ventricular pressure features from the ventricular hemodynamic data; derive, using a second algorithm, one or more pulmonary artery pressure features from the arterial hemodynamic data; derive, using a third algorithm, one or more cardiac output parameters from the ventricular hemodynamic data and/or the arterial hemodynamic data; derive, using a fourth algorithm, one or more venous oxygen saturation parameters from the blood oxygen saturation data; derive the GHI by using a predictive decision model based upon the one or more right ventricular pressure features, the one or more pulmonary artery features, the one or more cardiac output parameters, and the one or more venous oxygen saturation parameters; and display the GHI on the hemodynamic display.

The system of the preceding paragraph can optionally include, additionally and/or alternatively, any one or more of the following features, configurations and/or additional components:

The first algorithm is a right ventricular pressure algorithm.

The right ventricular pressure algorithm is applied to a right ventricular pressure waveform from the ventricular hemodynamic data to derive the one or more right ventricular pressure features.

The one or more right ventricular pressure features include one or more of: a pulse rate, a maximum pressure rate of change with respect to time (“dP/dt”) during systolic rise, a minimum dP/dt during a relaxation period after end systole, a systole time, a systolic pressure, an end systolic pressure, an end diastolic pressure, a pulse pressure, and a mean pressure.

The second algorithm is a pulmonary artery pressure (PAP) algorithm.

The PAP algorithm is applied to a pulmonary artery pressure waveform from the arterial hemodynamic data to derive the one or more pulmonary artery pressure features.

The one or more pulmonary artery features include a mean pulmonary artery pressure.

The third algorithm is a right ventricular cardiac output (RVCO) algorithm.

The RVCO algorithm is applied to a right ventricular pressure (RVP) waveform from the ventricular hemodynamic data and/or the arterial hemodynamic data to derive the one or more cardiac output parameters.

The cardiac output parameters include a continuous cardiac output.

The RVCO algorithm includes applying a validation module to the RVP waveform to determine if the RVP waveform is valid.

The RVCO algorithm includes converting the right ventricular pressure waveform of the patient into a waveform of blood flow of the patient via a machine learning model.

The machine learning model is a deep-learning model that uses neural network architecture.

The machine learning model is an autoencoder model.

The RVCO algorithm includes filtering the waveform of blood flow to remove artifacts or physiological inaccuracies to yield a processed blood flow waveform.

Negative flow, flow values in the thousands, square-shaped flow, an inordinate increase in blood flow, or inordinately dissimilar flow per beat is removed from the waveform of blood flow to yield the processed blood flow waveform.

The RVCO algorithm includes generating a signal quality index of the processed blood flow waveform, the signal quality index indicative of the quality of the signal based upon the amount of artifacts or physiological inaccuracies removed from the waveform of blood flow.

The signal quality index is generated based upon analysis of a ten-second interval of the waveform of blood flow of the patient.

The signal quality index indicates whether the processed blood flow waveform is a valid processed blood flow waveform or an invalid processed blood flow waveform.

The valid processed blood flow waveform is used to measure the one or more cardiac output parameters.

The RVCO algorithm is applied to a right ventricular pressure (RVP) waveform, a pulmonary artery pressure (PAP) waveform, and an intermittent cardiac output from the arterial hemodynamic data and/or the ventricular hemodynamic data to derive the one or more cardiac output parameters.

The RVCO algorithm includes applying a validation module to the RVP waveform and to the PAP waveform to determine if the RVP waveform and the PAP waveform are valid.

The fourth algorithm is a venous oxygen saturation algorithm.

The venous oxygen saturation algorithm is used to derive a venous oxygen saturation value and a signal quality index from the blood oxygen saturation data.

The system memory is further encoded with instructions that, when executed by the system processor, cause the system to display a graph of change in the venous oxygen saturation value over time on the hemodynamic display.

The predictive decision model includes a machine learning model, a feature creation module, and a model heuristics module.

The machine learning model is a predictive risk model based upon a linear, weighted set of predictive features that have been identified as being predictive of a global hypoperfusion event.

The set of predictive features predictive of the global hypoperfusion event are identified using a regression model that minimizes loss.

The feature creation module computes one or more intermediate features based upon the one or more right ventricular pressure features, the one or more cardiac output parameters, the one or more pulmonary artery pressure features, and the one or more venous oxygen saturation parameters.

The one or more intermediate features comprise arterial elastance and pulmonary vascular resistance.

The model heuristics module performs a validation check on the one or more right ventricular pressure features, the one or more cardiac output parameters, the one or more pulmonary artery pressure features, and the one or more venous oxygen saturation parameters, to determine if the data is valid for use in the machine learning model.

The system memory is further encoded with instructions that, when executed by the system processor, cause the system to assign a risk level to the global hypoperfusion index based on one or more predetermined thresholds, wherein the risk level is indicative of a patient condition based upon the global hypoperfusion index.

The system memory is further encoded with instructions that, when executed by the system processor, cause the system to output a sensory alarm when the risk level indicates that a patient assessment is required.

The risk level indicating that patient assessment is recommended is output when a predicted venous oxygen saturation value is below 60.

The one or more predetermined thresholds are indicative of a stable condition, a careful watch condition, and a patient assessment recommended condition.

The system memory is further encoded with instructions that, when executed by the system processor, cause the system to provide secondary screening information indicative of one or more possible causes of the patient condition when the risk level indicates that a patient assessment is required.

The secondary screening information comprises information indicative of whether cardiac output is related to the patient condition.

The secondary screening information comprises information indicative of whether oxygen uptake is related to the patient condition.

The catheter is a pulmonary artery catheter coupled to the arterial blood pressure sensor, the ventricular blood pressure sensor, and the oximetry module.

The one or more cardiac output parameters are derived from data generated from a thermistor or thermal filament of the catheter.

The third algorithm is an Arterial Pressure Cardiac Output (APCO) algorithm.

A method for determining a global hypoperfusion index (GHI) representative of a prediction of a future global hypoperfusion event within a patient includes receiving arterial hemodynamic data from an arterial blood pressure sensor; receiving ventricular hemodynamic data from a ventricular blood pressure sensor; receive blood oxygen saturation data from an oximetry module; deriving, using a first algorithm, one or more right ventricular pressure features from the ventricular hemodynamic data; deriving, using a second algorithm, one or more pulmonary artery pressure features from the arterial hemodynamic data; deriving, using a third algorithm, one or more cardiac output parameters from the ventricular hemodynamic data and/or the arterial hemodynamic data; deriving, using a fourth algorithm, one or more venous oxygen saturation parameters from the blood oxygen saturation data; deriving the GHI by using a predictive decision model based upon the one or more right ventricular pressure features, the one or more pulmonary artery features, the one or more cardiac output parameters, and the one or more venous oxygen saturation parameters; and displaying the GHI on a hemodynamic display.

The method of the preceding paragraph can optionally include, additionally and/or alternatively, any one or more of the following features, configurations and/or additional components:

The first algorithm is a right ventricular pressure algorithm.

The right ventricular pressure algorithm is applied to a right ventricular pressure waveform from the ventricular hemodynamic data to derive the one or more right ventricular pressure features.

The one or more right ventricular pressure features include one or more of: a pulse rate, a maximum pressure rate of change with respect to time (“dP/dt”) during systolic rise, a minimum dP/dt during a relaxation period after end systole, a systole time, a systolic pressure, an end systolic pressure, an end diastolic pressure, a pulse pressure, and a mean pressure.

The second algorithm is a pulmonary artery pressure (PAP) algorithm.

The PAP algorithm is applied to a pulmonary artery pressure waveform from the arterial hemodynamic data to derive the one or more pulmonary artery pressure features.

The one or more pulmonary artery features include a mean pulmonary artery pressure.

The third algorithm is a right ventricular cardiac output (RVCO) algorithm.

The cardiac output parameters include a continuous cardiac output.

The fourth algorithm is a venous oxygen saturation algorithm.

The venous oxygen saturation algorithm is used to derive a venous oxygen saturation value and a signal quality index from the arterial hemodynamic data and/or the ventricular hemodynamic data.

The method further includes assigning a risk level to the global hypoperfusion index based on one or more predetermined thresholds, wherein the risk level is indicative of a patient condition based upon the global hypoperfusion index.

The method further includes outputting a sensory alarm when the risk level indicates that a patient assessment is required.

The risk level indicating that patient assessment is recommended is output when a predicted venous oxygen saturation value is below 60.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system for determining a global hypoperfusion index (GHI) representative of a prediction of a future global hypoperfusion event within a patient, the system comprising:
an arterial blood pressure sensor including a first housing, a first fluid input port connected via tubing to a first fluid source, a first catheter-side fluid port connected to a catheter inserted within an arterial system of a patient, first pressure transducer in communication with the first fluid source through the first catheter-side fluid port, and a first I/O cable in electrical communication with the first pressure transducer;
a ventricular blood pressure sensor including a second housing, a second fluid input port connected via tubing to a second fluid source, a second catheter-side fluid port connected to the catheter inserted within a ventricular system of a patient, second pressure transducer in communication with the second fluid source through the second catheter-side fluid port, and a second I/O cable in electrical communication with the second pressure transducer;
an oximetry module including an optical transmitter, an optical receiver, and an I/O cable in electrical communication with the optical transmitter and the optical receiver;
an integrate hardware unit including:
a system processor;
a system memory;
a display including a user interface; and
an analog-to-digital (ADC) converter;
wherein the system memory includes instructions that, when executed by the system processor, cause the system to perform the following steps:
receive arterial hemodynamic data from the arterial blood pressure sensor;
receive ventricular hemodynamic data from the ventricular blood pressure sensor;
receive blood oxygen saturation data from the oximetry module;
derive, using a first algorithm, one or more right ventricular pressure features from the ventricular hemodynamic data;
derive, using a second algorithm, one or more pulmonary artery pressure features from the arterial hemodynamic data;
derive, using a third algorithm, one or more cardiac output parameters from the ventricular hemodynamic data and/or the arterial hemodynamic data;
derive, using a fourth algorithm, one or more venous oxygen saturation parameters from the blood oxygen saturation data;
derive the GHI by using a predictive decision model based upon the one or more right ventricular pressure features, the one or more pulmonary artery features, the one or more cardiac output parameters, and the one or more venous oxygen saturation parameters; and
display the GHI on the hemodynamic display.

2. The system of claim 1, wherein the first algorithm is a right ventricular pressure algorithm that is applied to a right ventricular pressure waveform from the ventricular hemodynamic data to derive the one or more right ventricular pressure features, and wherein the one or more right ventricular pressure features include one or more of: a pulse rate, a maximum pressure rate of change with respect to time (“dP/dt”) during systolic rise, a minimum dP/dt during a relaxation period after end systole, a systole time, a systolic pressure, an end systolic pressure, an end diastolic pressure, a pulse pressure, and a mean pressure.

3. The system of claim 1, wherein the second algorithm is a pulmonary artery pressure (PAP) algorithm, wherein the PAP algorithm is applied to a pulmonary artery pressure waveform from the arterial hemodynamic data to derive the one or more pulmonary artery pressure features, and wherein the one or more pulmonary artery features include a mean pulmonary artery pressure.

4. The system of claim 1, wherein the third algorithm is a right ventricular cardiac output (RVCO) algorithm, wherein the RVCO algorithm is applied to a right ventricular pressure (RVP) waveform from the ventricular hemodynamic data and/or the arterial hemodynamic data to derive the one or more cardiac output parameters, and wherein the cardiac output parameters include a continuous cardiac output.

5. The system of claim 4, wherein the RVCO algorithm includes applying a validation module to the RVP waveform to determine if the RVP waveform is valid.

6. The system of claim 4, wherein the RVCO algorithm includes converting the right ventricular pressure waveform of the patient into a waveform of blood flow of the patient via a machine learning model, and wherein the RVCO algorithm includes filtering the waveform of blood flow to remove artifacts or physiological inaccuracies to yield a processed blood flow waveform.

7. The system of claim 1, wherein the RVCO algorithm is applied to a right ventricular pressure (RVP) waveform, a pulmonary artery pressure (PAP) waveform, and an intermittent cardiac output from the arterial hemodynamic data and/or the ventricular hemodynamic data to derive the one or more cardiac output parameters.

8. The system of claim 7, wherein the RVCO algorithm includes applying a validation module to the RVP waveform and to the PAP waveform to determine if the RVP waveform and the PAP waveform are valid.

9. The system of claim 1, wherein the fourth algorithm is a venous oxygen saturation algorithm, wherein the venous oxygen saturation algorithm is used to derive a venous oxygen saturation value and a signal quality index from the blood oxygen saturation data.

10. The system of claim 1, wherein the system memory is further encoded with instructions that, when executed by the system processor, cause the system to:
display a graph of change in the venous oxygen saturation value over time on the hemodynamic display.

11. The system of claim 1, wherein:
the predictive decision model includes a machine learning model, a feature creation module, and a model heuristics module;
the machine learning model is a predictive risk model based upon a linear, weighted set of predictive features that have been identified as being predictive of a global hypoperfusion event, wherein the set of predictive features predictive of the global hypoperfusion event are identified using a regression model that minimizes loss; and
the feature creation module computes one or more intermediate features based upon the one or more right ventricular pressure features, the one or more cardiac output parameters, the one or more pulmonary artery pressure features, and the one or more venous oxygen saturation parameters, wherein the one or more intermediate features comprise arterial elastance and pulmonary vascular resistance.

12. The system of claim 1, wherein system memory is further encoded with instructions that, when executed by the system processor, cause the system to:
assign a risk level to the global hypoperfusion index based on one or more predetermined thresholds, wherein the risk level is indicative of a patient condition based upon the global hypoperfusion index; and
output a sensory alarm when the risk level indicates that a patient assessment is required, wherein the risk level indicating that patient assessment is recommended is output when a predicted venous oxygen saturation value is below 60.

13. The system of claim 12, wherein the system memory is further encoded with instructions that, when executed by the system processor, cause the system to:
provide secondary screening information indicative of one or more possible causes of the patient condition when the risk level indicates that a patient assessment is required.

14. The system of claim 1, wherein the third algorithm is an Arterial Pressure Cardiac Output (APCO) algorithm.

15. A method for determining a global hypoperfusion index (GHI) representative of a prediction of a future global hypoperfusion event within a patient, the method comprising:
receiving arterial hemodynamic data from an arterial blood pressure sensor;
receiving ventricular hemodynamic data from a ventricular blood pressure sensor;
receive blood oxygen saturation data from an oximetry module;
deriving, using a right ventricular pressure algorithm, one or more right ventricular pressure features from the ventricular hemodynamic data;
deriving, using a pulmonary artery pressure (PAP) algorithm, one or more pulmonary artery pressure features from the arterial hemodynamic data;
deriving, using a right ventricular cardiac output (RVCO) algorithm, one or more cardiac output parameters from the ventricular hemodynamic data and/or the arterial hemodynamic data;
deriving, using a venous oxygen saturation algorithm, one or more venous oxygen saturation parameters from the blood oxygen saturation data;
deriving the GHI by using a predictive decision model based upon the one or more right ventricular pressure features, the one or more pulmonary artery features, the one or more cardiac output parameters, and the one or more venous oxygen saturation parameters; and
displaying the GHI on a hemodynamic display.

16. The method of claim 15, wherein the right ventricular pressure algorithm is applied to a right ventricular pressure waveform from the ventricular hemodynamic data to derive the one or more right ventricular pressure features, and wherein the one or more right ventricular pressure features include one or more of: a pulse rate, a maximum pressure rate of change with respect to time (“dP/dt”) during systolic rise, a minimum dP/dt during a relaxation period after end systole, a systole time, a systolic pressure, an end systolic pressure, an end diastolic pressure, a pulse pressure, and a mean pressure.

17. The method of claim 15, wherein the PAP algorithm is applied to a pulmonary artery pressure waveform from the arterial hemodynamic data to derive the one or more pulmonary artery pressure features, and wherein the one or more pulmonary artery features include a mean pulmonary artery pressure.

18. The method of claim 15, wherein the cardiac output parameters include a continuous cardiac output.

19. The method of claim 15, wherein the venous oxygen saturation algorithm is used to derive a venous oxygen saturation value and a signal quality index from the arterial hemodynamic data and/or the ventricular hemodynamic data.

20. The method of claim 15, further comprising:
assigning a risk level to the global hypoperfusion index based on one or more predetermined thresholds, wherein the risk level is indicative of a patient condition based upon the global hypoperfusion index; and
outputting a sensory alarm when the risk level indicates that a patient assessment is required, wherein the risk level indicating that patient assessment is recommended is output when a predicted venous oxygen saturation value is below 60.
